(19) United States
(12) Patent Application Publication
(10) Pub. No.: US 2020/0178981 A1
(43) Pub. Date: Jun. 11, 2020

(54) LEFT ATRIAL APPENDAGE IMPLANT WITH SEALING BALLOON

(71) Applicant: BOSTON SCIENTIFIC SCIMED, INC., MAPLE GROVE, MN (US)

(72) Inventors: James M. Anderson, Corcoran, MN (US); Brian Joseph Tischler, Shoreview, MN (US); Joshua Mark Inouye, Maple Grove, MN (US); David John Onushko, Maple Grove, MN (US)

(73) Assignee: BOSTON SCIENTIFIC SCIMED, INC., MAPLE GROVE, MN (US)

(21) Appl. No.: 16/707,709

(22) Filed: Dec. 9, 2019

Related U.S. Application Data
(60) Provisional application No. 62/777,495, filed on Dec. 10, 2018.

(51) Int. Cl. A61B 17/12

(52) U.S. Cl. CPC A61B 17/12122; A61B 2017/00632; A61B 17/12031; A61B 17/12172

(57) ABSTRACT

An implant for occluding a left atrial appendage may include an expandable framework configured to shift between a collapsed configuration and an expanded configuration, an occlusive element disposed on the expandable framework, and a sealing member spaced apart proximally from the expandable framework by a gap distance. A system for occluding a left atrial appendage may further include a delivery sheath and a core wire releasably secured to the implant. A method for occluding a left atrial appendage may include advancing the implant to the left atrial appendage, deploying the expandable framework within the left atrial appendage, shifting the expandable framework into the expanded configuration within the left atrial appendage, and deploying the sealing member proximate an ostium of the left atrial appendage.

### CROSS-REFERENCE TO RELATED APPLICATIONS

This application claims the benefit of priority under 35 U.S.C. § 119 to U.S. Provisional Application Ser. No. 62/777,495, filed Dec. 10, 2018, the entirety of which is incorporated herein by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

The occurrence of thrombi in the left atrial appendage during atrial fibrillation may be due to stagnancy of the blood pool in the left atrial appendage. The blood may still be pulled out of the left atrium by the left ventricle, however less effectively due to the irregular contraction of the left atrium caused by atrial fibrillation. Therefore, instead of an active support of the blood flow by a contracting left atrium and left atrial appendage, filling of the left ventricle may depend primarily or solely on the suction effect created by the left ventricle. Further, the contraction of the left atrial appendage may not be in sync with the cycle of the left ventricle. For example, contraction of the left atrial appendage may be out of phase up to 180 degrees with the left ventricle, which may create significant resistance to the desired flow of blood. Further still, most left atrial appendage geometries are complex with large irregular surface areas and a narrow ostium or opening compared to the depth of the left atrial appendage. These aspects as well as others, taken individually or in various combinations, may lead to high flow resistance of blood out of the left atrial appendage and/or formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Over time, exposed surface(s) of an implant spanning the left atrial appendage may become covered with tissue (a process called endothelization), effectively removing the left atrial appendage from the circulatory system and reducing or eliminating the amount of thrombi which may enter the blood stream from the left atrial appendage. Of the known medical devices and methods, each has certain advantages and disadvantages. There is an ongoing need to provide alternative medical devices and introducers as well as alternative methods for manufacturing and using medical devices and introducers.

### SUMMARY

In a first aspect, an implant for occluding a left atrial appendage may comprise an expandable framework configured to shift between a collapsed configuration and an expanded configuration, and a sealing member spaced apart proximally from the expandable framework by a gap distance.

In addition or alternatively, and in a second aspect, the sealing member is connected to the expandable framework by a flexible coupler.

In addition or alternatively, and in a third aspect, the flexible coupler is tubular.

In addition or alternatively, and in a fourth aspect, the gap distance is variable.

In addition or alternatively, and in a fifth aspect, the implant may further comprise a tapered member configured to vary the gap distance.

In addition or alternatively, and in a sixth aspect, the implant may further comprise a threaded adjustment configured to vary the gap distance.

In addition or alternatively, and in a seventh aspect, the threaded adjustment couples the sealing member to the expandable framework.

In addition or alternatively, and in an eighth aspect, the sealing member includes an inflatable disk-shaped member.

In addition or alternatively, and in a ninth aspect, the sealing member includes an inflatable annular member defining a central space.

In addition or alternatively, and in a tenth aspect, the sealing member includes a first layer extending across the central space and a second layer extending across the central space.

In addition or alternatively, and in an eleventh aspect, the first layer is spaced apart from the second layer.

In addition or alternatively, and in a twelfth aspect, at least one of the first layer or the second layer includes a plurality of reinforcing fibers.

In addition or alternatively, and in a thirteenth aspect, a system for occluding a left atrial appendage may comprise a delivery sheath having a lumen; an implant for occluding the left atrial appendage, the implant comprising: an expandable framework configured to shift between a collapsed configuration and an expanded configuration, and a sealing member connected to and spaced apart proximally from the expandable framework by a flexible coupler; and a core wire releasably secured to the implant.

In addition or alternatively, and in a fourteenth aspect, the system may further comprise an inflation lumen in fluid communication with the sealing member.

In addition or alternatively, and in a fifteenth aspect, the inflation lumen extends through the core wire.

In addition or alternatively, and in a sixteenth aspect, a method for occluding a left atrial appendage may comprise:

advancing an implant to the left atrial appendage, the implant including:
an expandable framework configured to shift between a collapsed configuration and an expanded configuration; and
a sealing member spaced apart proximally from the expandable framework by a gap distance;

deploying the expandable framework within the left atrial appendage;

shifting the expandable framework into the expanded configuration within the left atrial appendage; and

deploying the sealing member proximate an ostium of the left atrial appendage.

In addition or alternatively, and in a seventeenth aspect, the method may further comprise inflating at least a portion of the sealing member until the sealing member is capable of engaging the ostium in a sealing manner.

In addition or alternatively, and in an eighteenth aspect, the method may further comprise adjusting the gap distance to position the sealing member against or within the ostium.

In addition or alternatively, and in a nineteenth aspect, the sealing member is oriented at an oblique angle to a central longitudinal axis of the expandable framework.

In addition or alternatively, and in a twentieth aspect, the sealing member includes a mesh configured to promote endothelization.

The above summary of some embodiments, aspects, and/or examples is not intended to describe each embodiment or every implementation of the present disclosure.

The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic partial cross-sectional view of a heart;

FIG. 2 is a schematic partial cross-sectional view of an example left atrial appendage;

FIGS. 3-5 illustrate aspects of a system, implant, and/or method for occluding a left atrial appendage;

FIGS. 6-8 illustrate aspects of a means for adjusting a gap distance between an example expandable framework and an example sealing member;

FIG. 9 illustrates aspects of an alternative means for adjusting a gap distance between an example expandable framework and an example sealing member;

FIGS. 10-11 are cross-sectional views of selected portions of FIG. 9;

FIG. 12 illustrates aspects of an alternative sealing member;

FIGS. 13A-13C are cross-sectional views illustrating alternative configurations of the sealing member of FIG. 12;

FIGS. 14-18 illustrate aspects of systems, implants, and/or methods for occluding a left atrial appendage; and

FIG. 19 illustrates an example implant of the disclosure disposed within a left atrial appendage having an irregular configuration.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

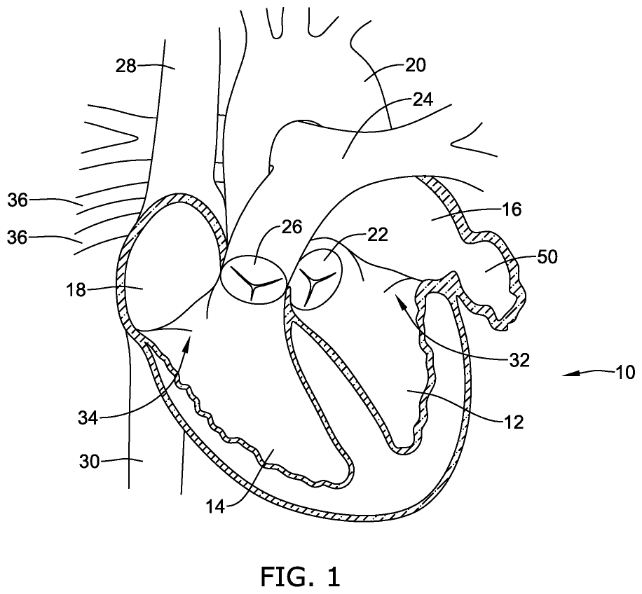

FIG. 1 is a partial cross-sectional view of certain elements of a human heart 10 and some selected adjacent blood vessels. A heart 10 may include a left ventricle 12, a right ventricle 14, a left atrium 16, and a right atrium 18. An aortic valve 22 is disposed between the left ventricle 12 and an aorta 20. A pulmonary or semi-lunar valve 26 is disposed between the right ventricle 14 and a pulmonary artery 24. A superior vena cava 28 and an inferior vena cava 30 return blood from the body to the right atrium 18. A mitral valve 32 is disposed between the left atrium 16 and the left ventricle 12. A tricuspid valve 34 is disposed between the right atrium 18 and the right ventricle 14. Pulmonary veins 36 return blood from the lungs to the left atrium 16. A left atrial appendage 50 is attached to and in fluid communication with the left atrium 16.

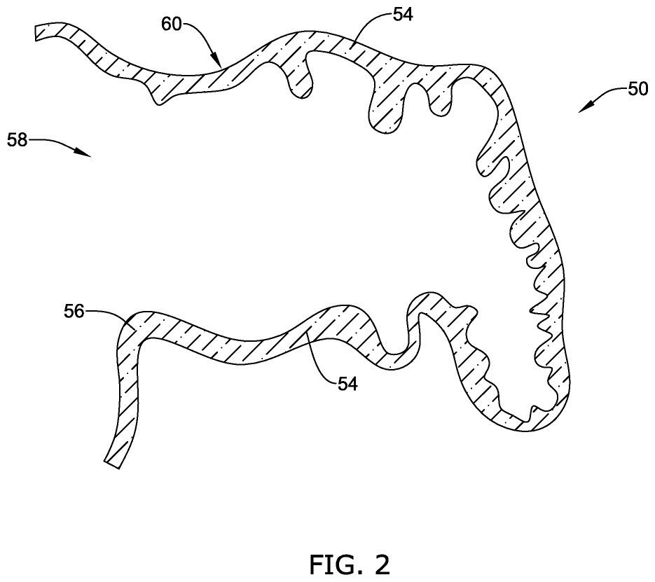

FIG. 2 is a partial cross-sectional view of an example left atrial appendage 50. As discussed above, the left atrial appendage 50 may have a complex geometry and/or irregular surface area. Those skilled in the art will recognize that the illustrated left atrial appendage is merely one of many possible shapes and sizes for the left atrial appendage, which may vary from patient to patient. Those of skill in the art will also recognize that the medical devices and methods disclosed herein may be adapted for various sizes and shapes of the left atrial appendage, as necessary. A left atrial appendage 50 may include a generally longitudinal axis arranged along a depth of a main body 60 of the left atrial appendage 50. The main body 60 may include a wall 54 and an ostium 56 forming a proximal mouth 58. In some embodiments, a lateral extent of the ostium 56 and/or the wall 54 may be smaller or less than a depth of the main body 60 along the longitudinal axis, or a depth of the main body 60 may be greater than a lateral extent of the ostium 56 and/or the wall 54. In some embodiments, the left atrial appendage 50 may include a tail-like element associated with a distal portion of the main body 60, which element may protrude radially or laterally away from the main body 60.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

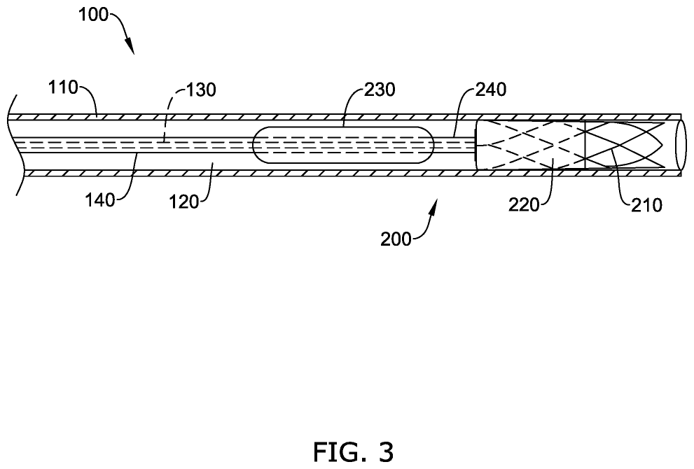

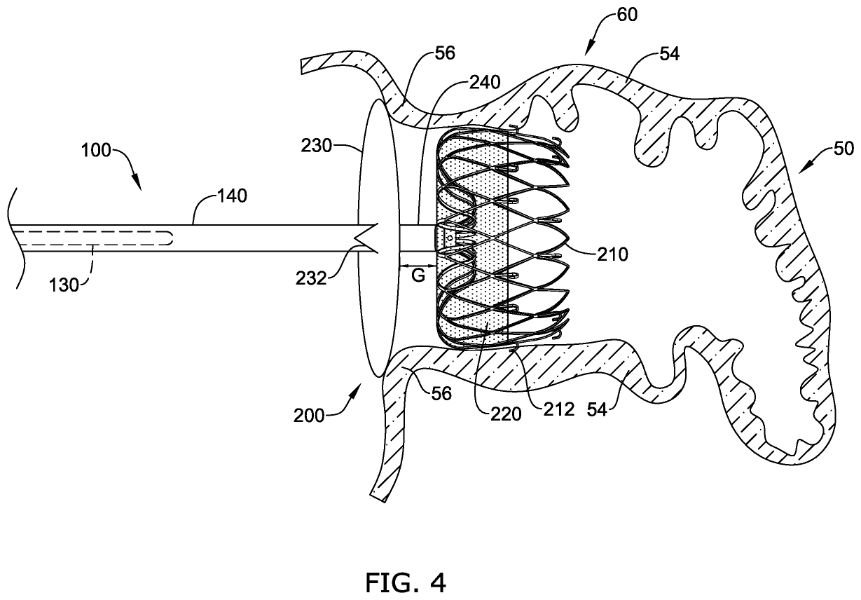

FIG. 3 is a partial cross-sectional view illustrating elements of a system 100 for occluding the left atrial appendage 50. The system 100 may include a delivery sheath 110 having a lumen 120 extending to a distal end. The system 100 may include an implant 200 for occluding the left atrial appendage 50. The implant 200 may comprise an expandable framework 210 configured to shift between a collapsed configuration and an expanded configuration. When the implant 200 is disposed within the lumen 120 of the delivery sheath 110, the expandable framework 210 may be held and/or disposed in the collapsed configuration, as shown in FIG. 3 for example. In some embodiments, the implant 200 may optionally include an occlusive element 220 disposed and/or positioned on, over, and/or around at least a portion of the expandable framework 210. In at least some embodiments, the occlusive element 220 may be secured to, attached to, and/or connected to the expandable framework 210. In some embodiments, the occlusive element 220 may be secured to, attached to, and/or connected to the expandable framework 210 at a plurality of discrete locations. In at least some embodiments, the expandable framework 210 may include a plurality of anchor members 212 (e.g., FIG. 4) extending therefrom, the plurality of anchor members 212 being configured to engage with the wall 54 of the main body 60 of the left atrial appendage 50. Some suitable, but non-limiting, examples of materials for the delivery sheath 110, the expandable framework 210, the plurality of anchor members 212, and the occlusive element 220 are discussed below.

The implant 200 may comprise a sealing member 230 spaced apart proximally from the expandable framework 210 by a flexible coupler 240. In at least some embodiments, the sealing member 230 may be secured to, attached to, and/or connected to the expandable framework 210 by the flexible coupler 240. In some embodiments, the flexible coupler 240 is tubular (e.g., a tubular member, a hollow tube, etc.) and includes a lumen extending therethrough. In some embodiments, the flexible coupler 240 may be formed by one or more filaments or sutures, one or more flexible members spaced apart from each other, a discontinuous flexible element having notches or cut-outs formed therein, a coiled member, or other suitable flexible structures. In at least some embodiments, the sealing member 230 may be at least partially inflatable. The sealing member 230 may be configured to shift between a delivery configuration and a deployed configuration. When the implant 200 and/or the sealing member 230 is disposed within the lumen 120 of the delivery sheath 110, the sealing member 230 may be held and/or disposed in the delivery configuration. In some embodiments, the sealing member 230 may include a mesh, a fabric, or other surface treatment configured to promote endothelization on and/or across the sealing member 230. In some embodiments, the sealing member 230 may include the mesh, the fabric, or the other surface treatment disposed on and/or surrounding a portion of an outer surface of the sealing member 230. In some embodiments, the sealing member 230 may include the mesh, the fabric, or the other surface treatment disposed on and/or surrounding an entire outer surface of the sealing member 230. In some embodiments, the mesh, the fabric, or the other surface treatment may be elastic and/or stretchable to accommodate changes in shape and/or size of the sealing member 230 when the sealing member 230 is shifted toward and/or into the deployed configuration. Some suitable, but non-limiting, examples of materials for the sealing member 230 and the flexible coupler 240 are discussed below.

In some embodiments, the system 100 may include a core wire 130 releasably secured and/or releasably connected to the implant 200 at a distal end of the core wire 130. In some embodiments, the core wire 130 may be engaged with, releasably secured to, and/or releasably connected to the expandable framework 210 or the sealing member 230. In some embodiments, wherein the core wire 130 is engaged with, releasably secured to, and/or releasably connected to the expandable framework 210, the core wire 130 may pass through the sealing member 230. For example, the core wire 130 may pass through a self-sealing port and/or an aperture extending through the sealing member 230. In some embodiments, the core wire 130 may extend through the flexible coupler 240 to engage with the expandable framework 210. In some embodiments, the core wire 130 may engage with the sealing member 230 and/or the flexible coupler 240. Some suitable, but non-limiting, examples of materials for the core wire 130 are discussed below.

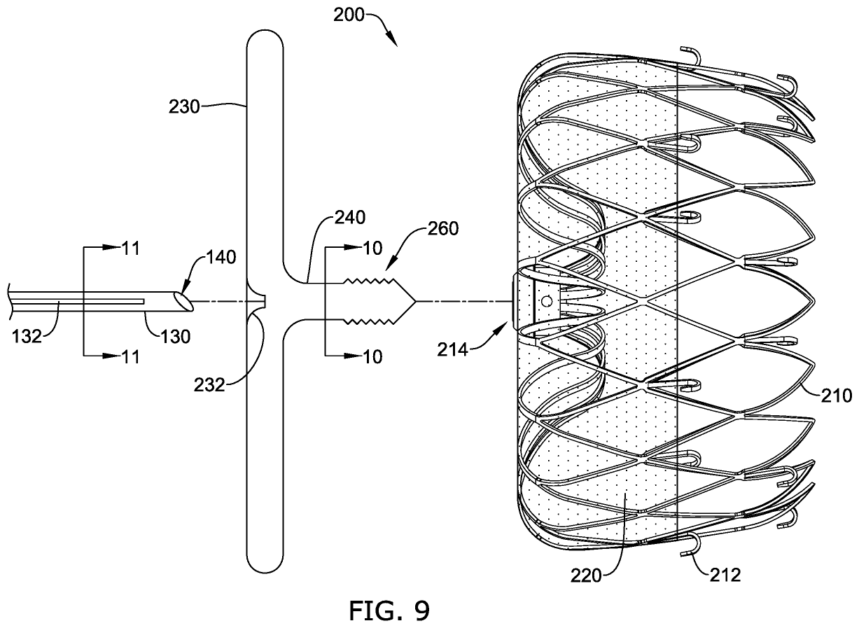

In some embodiments, the system 100 may include an inflation lumen 140 in fluid communication with the sealing member 230. The inflation lumen 140 may extend through the lumen 120 of the delivery sheath 110 to the sealing member 230. In some embodiments, the inflation lumen 140 may extend through the core wire 130 (e.g., FIG. 9). The sealing member 230 may be expandable under internal pressure exerted by an inflation fluid. In some embodiments, the inflation fluid may include a contrast agent for improved visualization under fluoroscopy. In some embodiments, the inflation fluid may be and/or include a hardening agent and/or a hardening or semi-hardening fluid. For example, the inflation fluid may include a biocompatible liquid such as saline, a hydropolymer, a hydrogel, or other suitable fluids. In at least some embodiments, an external shape of the sealing member 230 may be compliant, flexible, and/or adaptable to its surroundings. For example, the external shape of the sealing member 230 may shift and/or adapt to match the wall 54 and/or the ostium 56 of the left atrial appendage 50 disposed adjacent to the sealing member 230 upon implantation and thereby engage the wall 54 and/or the ostium 56 of the left atrial appendage 50 in a sealing manner.

A method for occluding the left atrial appendage 50 may comprise advancing the implant 200 to the left atrial appendage 50. For example, the implant 200 may be advanced to the left atrial appendage within the lumen 120 of the delivery sheath 110. The method includes deploying the expandable framework 210 from the delivery sheath 110 within the left atrial appendage 50. The method further includes expanding and/or shifting the expandable framework 210 from the collapsed configuration to the expanded configuration within the left atrial appendage 50, as seen in FIG. 4 for example. In the expanded configuration, the expandable framework 210 may be urged into contact with, engaged with, and/or anchored to the wall 54 of the main body 60 of the left atrial appendage 50. Additionally, the method may include deploying the sealing member 230 proximate the ostium 56 of the left atrial appendage 50. In some embodiments, the sealing member 230 may be spaced apart proximally from the expandable framework 210 by a gap distance G. The gap distance G may be generally understood as the axial distance between a proximal surface of the expandable framework 210 and a distal surface of the sealing member 230 measured generally parallel to a central longitudinal axis of the implant 200, the expandable framework 210, and/or the sealing member 230. In some embodiments, the gap distance G may be fixed. In some embodiments, the gap distance G may be variable. The expandable framework 210 and/or the plurality of anchor members 212 may function as an anchoring mechanism for the sealing member 230.

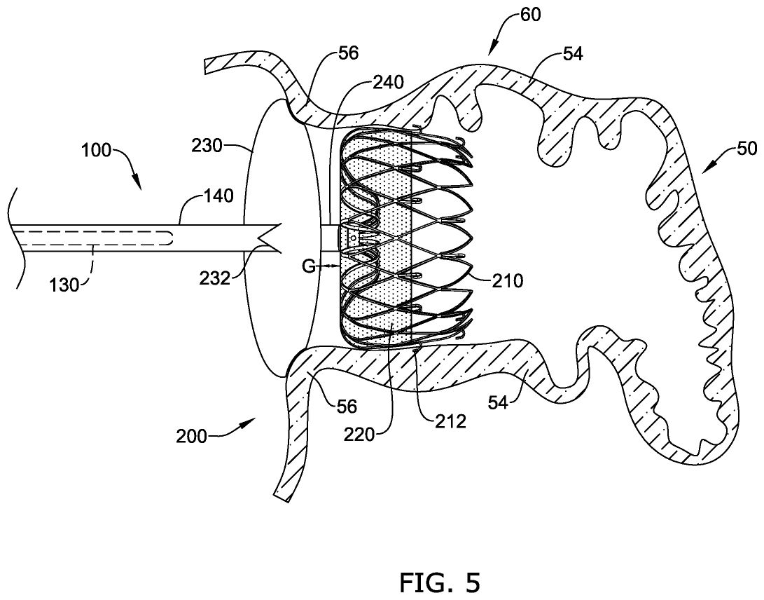

In some embodiments, the sealing member 230 may include an inflation port 232 configured to accept and/or engage with the inflation lumen 140. In some embodiments, the inflation port 232 may be a self-sealing port, and/or may include a hemostasis valve or other feature configured to seal the inflation port 232 in the absence of a structure (e.g., the inflation lumen 140, the core wire 130, etc.) disposed within the inflation port 232. In some embodiments, the core wire 130 may engage with and/or pass through the inflation port 232. In at least some embodiments, the sealing member 230 may be compliant and/or adaptable to its surroundings. As noted herein, the sealing member 230 may shift and/or adapt to fit and/or match the contour of the wall 54 and/or the ostium 56 of the left atrial appendage 50 disposed adjacent to the sealing member 230 upon implantation and thereby engage the wall 54 and/or the ostium 56 of the left atrial appendage 50 in a sealing manner. In at least some embodiments, the method may further include inflating at least a portion of the sealing member 230 until the sealing member 230 is capable of engaging the ostium 56 of the left atrial appendage 50 in a sealing manner, as seen in FIG. 5 for example.

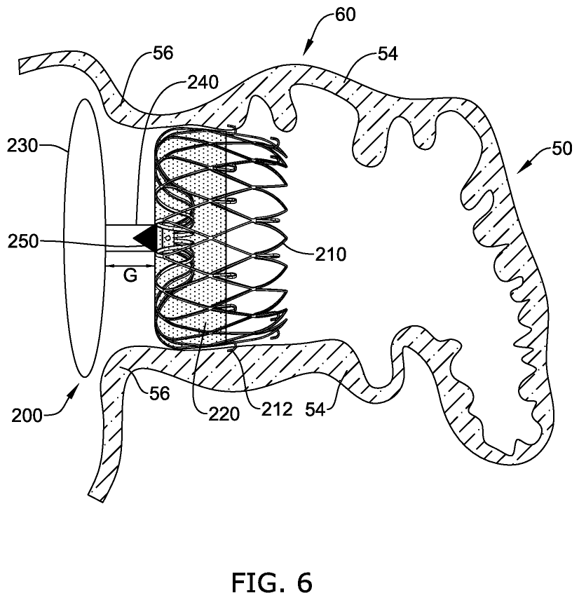

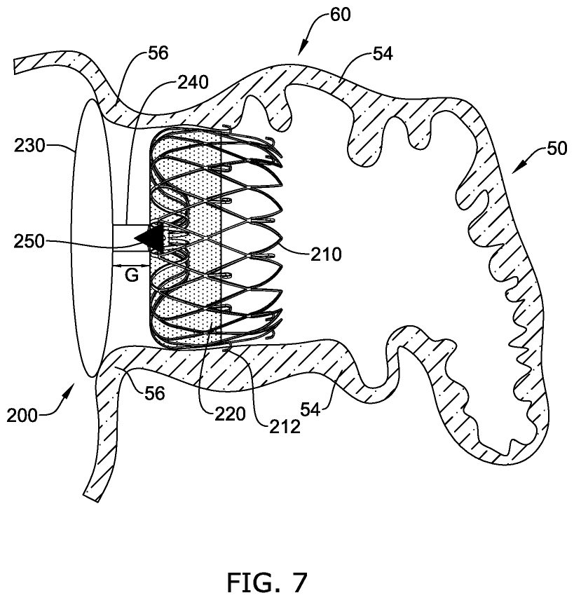

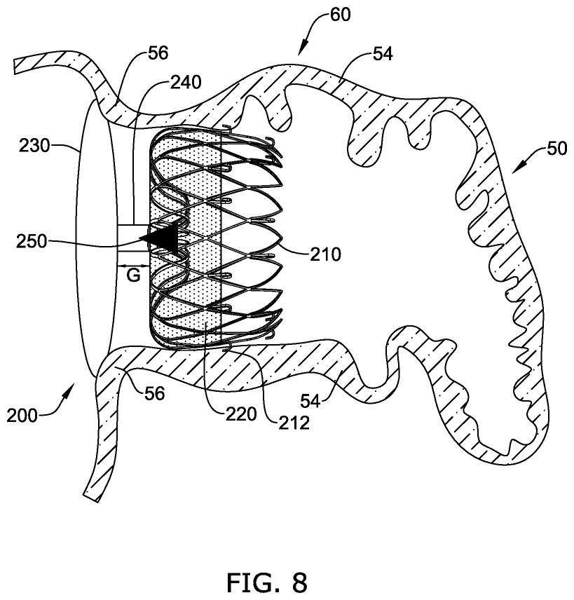

The system 100 and/or the implant 200 may include at least one means of adjusting the gap distance G. In some embodiments, the method for occluding the left atrial appendage 50 may comprise adjusting the gap distance G to position the sealing member 230 against and/or within the ostium 56 of the left atrial appendage 50. In some embodiments, the at least one means of adjusting the gap distance G may be configured to translate the sealing member 230 towards and/or into the ostium 56 of the left atrial appendage 50. For example, upon initial deployment of the implant 200, the expandable framework 210 may urged into contact with, engaged with, and/or anchored to the wall 54 of the main body 60 of the left atrial appendage 50, and the sealing member 230 may be spaced apart from the ostium 56 of the left atrial appendage 50, as seen in FIG. 6 for example. The at least one means of adjusting the gap distance G may be used to translate the sealing member 230 towards and/or into the ostium 56 of the left atrial appendage 50, as in FIGS. 7 and 8.

In one example, the implant 200 may include a tapered member 250 configured to vary the gap distance G, as shown in FIGS. 6-8. The tapered member 250 may be secured to and/or connected to the flexible coupler 240. In some embodiments, the tapered member 250 may be fixedly secured to and/or fixedly connected to a distal end of the flexible coupler 240. In some embodiments, a proximal-facing surface of the tapered member 250 may engage with and/or be in contact with the expandable framework 210. In some embodiments, the tapered member 250 may be prevented from translating through and/or proximal of the expandable framework 210. In at least some embodiments, the tapered member 250 may be an inflatable tapered member configured to expand axially and/or radially/laterally, wherein different degrees and/or magnitudes of inflation determine adjustment of the gap distance G. For example, as the tapered member 250 is inflated, the distal end of the flexible coupler 240 may be translated and/or pulled distally through and/or relative to the expandable framework 210, thereby shortening and/or reducing the gap distance G, and translating the sealing member 230 towards and/or into the ostium 56 of the left atrial appendage 50, as shown in FIGS. 7 and 8 for example. In one example, the gap distance G may be shortened by about 10%, about 20%, about 25%, about 30%, about 40%, about 50%, about 60%, about 75%, etc. from its initial deployment distance. In another example, the gap distance G may be shortened or reduced to zero. In some embodiments, the gap distance G may be shortened or reduced until the sealing member 230 makes contact with the tapered member 250.

In another example, the implant 200 may include a threaded adjustment configured to vary the gap distance G. In some embodiments, the sealing member 230 and/or the flexible coupler 240 may include a threaded portion 260 configured to rotatably and/or threadably engage with a corresponding and/or complimentary threaded portion 214 of the expandable framework 210, as seen in FIG. 9 for example. In some embodiments, the threaded adjustment (and/or the threaded portions 260/214) couples the sealing member 230 to the expandable framework 210. In some embodiments, the sealing member 230 and the flexible coupler 240 may be integrally formed as a unitary structure. In some embodiments, the sealing member 230 may include an inflatable disk-shaped member. In some embodiments, the sealing member 230 may include the inflatable disk-shaped member and an axial stem extending longitudinally away from the inflatable disk-shaped member. For example, in some embodiments, the sealing member 230 may have a form or shape similar to a flat-capped mushroom, which form or shape may be a generally flattened cap or head rather than a rounded or bulbous cap or head. In some embodiments, the sealing member 230 may have a regular or irregular surface or shape, a smooth or uneven surface or shape, and/or a concave or convex surface or shape. The sealing member 230 may be inflatable to engage the wall 54 and/or the ostium 56 of the left atrial appendage 50 in a sealing manner.

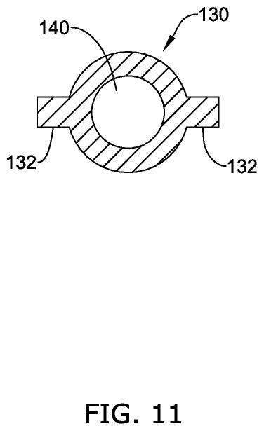

In the example shown in FIG. 9, a distal portion of the core wire 130 may include an external keying structure 132 (e.g., FIG. 11) configured to non-rotatably engage with the sealing member 230, the flexible coupler 240, and/or the threaded portion 260. The core wire 130 may be hollow and/or tubular, having the inflation lumen 140 extending therethrough. The sealing member 230 may include the inflation port 232 disposed proximate a proximal portion and/or a proximal end of the sealing member 230. The distal portion of the core wire 130 may be configured to extend through the inflation port 232 and into the sealing member 230. Injection of inflation fluid through the inflation lumen 140 and/or the core wire 130, while a distal end of the core wire 130 is disposed within the sealing member 230, may expand and/or inflate the sealing member 230 to engage the wall 54 and/or the ostium 56 of the left atrial appendage 50 in a sealing manner.

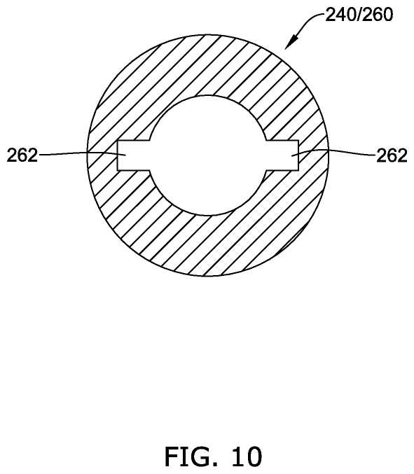

The core wire 130 may be configured to further extend and/or advance distally to bring the external keying structure 132 into engagement with an internal keying feature 262 (e.g., FIG. 10) within the flexible coupler 240 and/or the threaded portion 260. When the external keying structure 132 is engaged with the internal keying feature 262, rotation of the core wire 130 may be transmitted to the sealing member 230, the flexible coupler 240, and/or the threaded portion 260. Rotation of the sealing member 230, the flexible coupler 240, and/or the threaded portion 260 relative to the expandable framework 210 and/or the threaded portion 214 may vary the gap distance G by translating the sealing member 230 closer to or farther from the expandable framework 210 in response to the threaded engagement. In some embodiments, the distal end of the core wire 130 (and/or another structure or feature) may be extendable through the flexible coupler 240 and/or the threaded portion 260. For example, a guidewire may be positionable within the lumen of the core wire 130 for navigation of the implant 200 through the patient's vasculature and/or to the left atrial appendage 50. Accordingly, the distal end of the flexible coupler 240 and/or the threaded portion 260 may be self-sealing, and/or may include a hemostasis valve or other feature, such that withdrawal of the distal end of the core wire 130 therethrough permits the sealing member 230 to retain inflation fluid therein.

In another example, the flexible coupler 240 may include and/or be formed by one or more filaments, sutures, or other flexible elements. The means for adjusting the gap distance G may include shortening the flexible coupler 240. For example, the one or more filaments, sutures, or other flexible elements may be pulled through a cinch or latching feature, tied into one or more knots, twisted together, or other methods of shortening or taking up slack in the flexible coupler 240. Other configurations and/or arrangements are also contemplated.

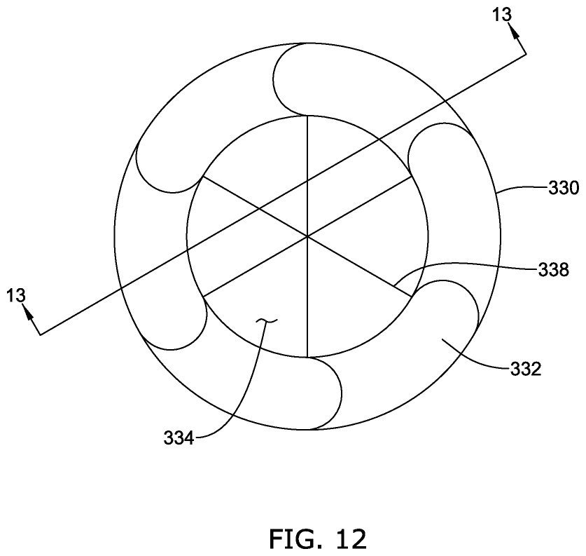

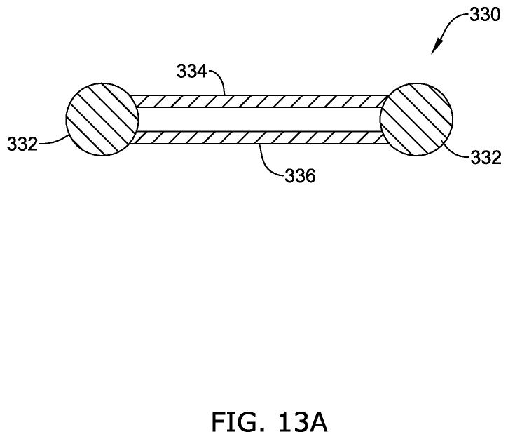

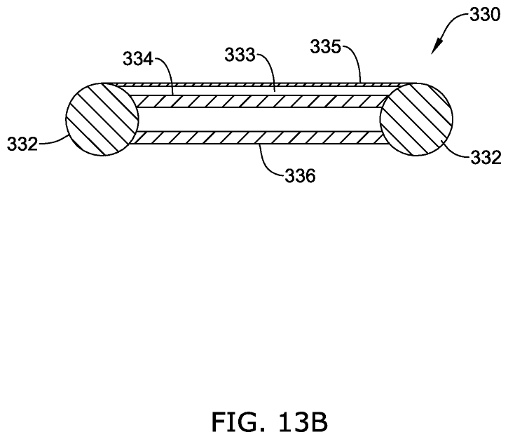

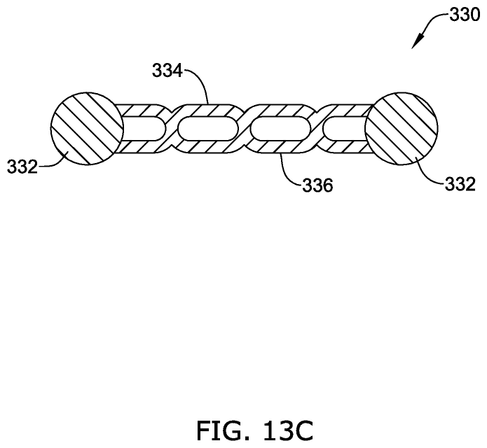

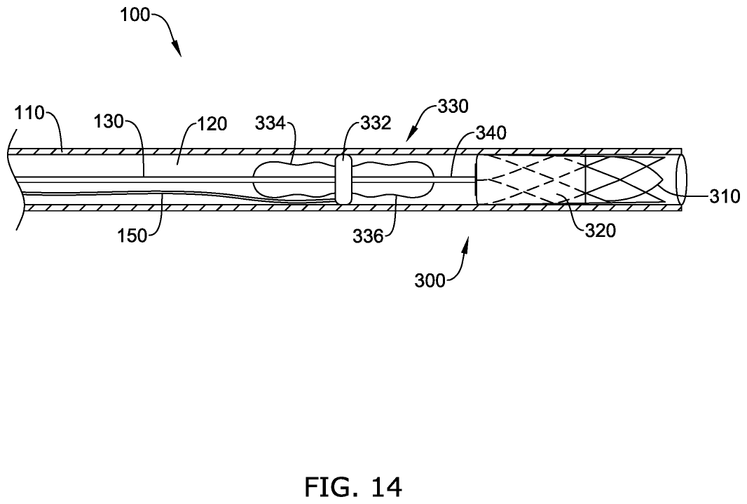

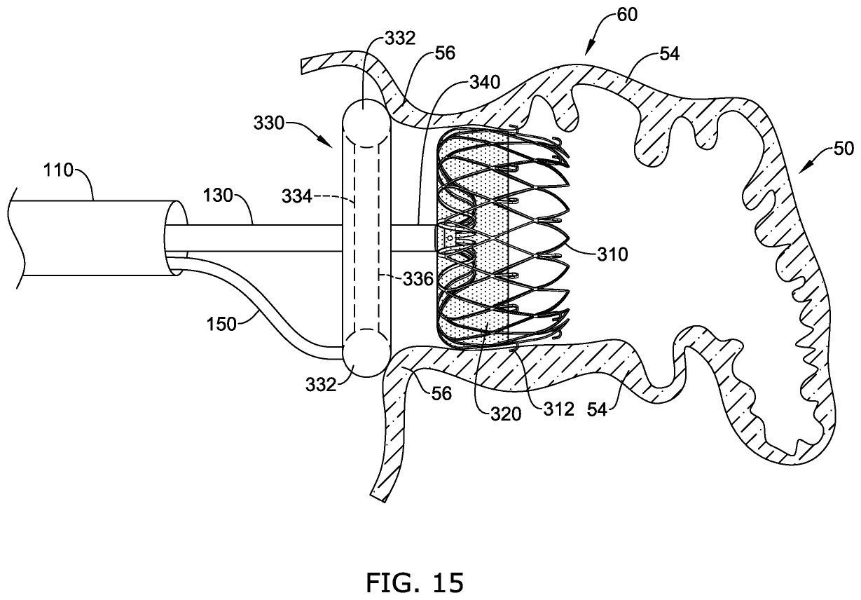

In an alternative embodiment, an implant 300 (e.g., FIGS. 14-15) may include a sealing member 330 having an inflatable annular member 332, as seen in FIG. 12 for example, the inflatable annular member 332 defining a central space. The inflatable annular member 332 may be formed from a polymeric material, a metallic material, and/or a composite material. In some embodiments, the inflatable annular member 332 may be formed from a substantially compliant material. In some other embodiments, the inflatable annular member 332 may be formed from a substantially non-compliant material. The sealing member 330 may include a first layer 334 extending across the central space and a second layer 336 extending across the central space, as seen in FIGS. 13A-13C. In some embodiments, the first layer 334 may be spaced apart from the second layer 336 across at least a portion of the central space. In some embodiments, the first layer 334 may be spaced apart from the second layer 336 across the entire central space (e.g., FIG. 13A). In some embodiments, the sealing member 330 may include a mesh 335, a fabric, or other surface treatment configured to promote endothelization on and/or across the sealing member 330. In some embodiments, the sealing member 330 may include the mesh 335, the fabric, or the other surface treatment disposed on and/or surrounding a portion of an outer surface of the sealing member 330. In some embodiments, the sealing member 330 may include the mesh 335, the fabric, or the other surface treatment disposed on and/or surrounding an entire outer surface of the sealing member 330. In some embodiments, the mesh 335, the fabric, or the other surface treatment may be elastic and/or stretchable to accommodate changes in shape and/or size of the sealing member 330 when the sealing member 330 is shifted toward and/or into the deployed configuration. In some embodiments, the mesh 335, the fabric, or the other surface treatment may be spaced apart from the first layer 334 and/or the second layer 336 (e.g., FIG. 13B). For example, a space 333 may be formed between the mesh 335, the fabric, or the other surface treatment and the first layer 334 and/or the second layer 336. The sealing member 330 may be configured to promote formation of organized microthrombus to enhance endothelial migration and coverage. The space 333 may be configured to cause stasis and promote coagulation, even in fully anti-coagulated patients, thereby creating a captive thrombus that may provide a medium for endothelial growth on the mesh 335, the fabric, or the other surface treatment. Additionally, the captive thrombus and/or endothelial growth on the mesh 335, the fabric, or the other surface treatment may provide a substrate for an external thrombus to adhere to, thereby preventing dislocation and/or embolization of the external thrombus. In some embodiments, the space 333 be formed with a distance of between 1% and 50% of an overall thickness of the sealing member 330 and/or the inflatable annular member 332. Other arrangements and/or configurations are also contemplated. In some embodiments, the first layer 334 may be spaced apart from the second layer 336 across at least a portion of the central space, and the first layer 334 may be discontinuously and/or intermittently secured, connected, and/or bonded to the second layer 336 at one or more discrete locations (e.g., FIG. 13C).

In some embodiments, at least one of the first layer 334 or the second layer 336 may include a plurality of reinforcing fibers 338, as shown in FIG. 12. In some embodiments, the plurality of reinforcing fibers 338 may include individual filaments, fabrics or textiles, mesh, or other suitable reinforcing elements. Other configurations and/or arrangements are also contemplated. The plurality of reinforcing fibers 338 may prevent stretching of the first layer 334 and/or the second layer 336 as the sealing member 330 and/or the inflatable annular member 332 is inflated and/or expanded. In some embodiments, the mesh 335, the fabric, or the other surface treatment may be inelastic and/or non-compliant to prevent changes in shape and/or size of the sealing member 330, the first layer 334, and/or the second layer 336 when the sealing member 330 is shifted toward and/or into the deployed configuration. In some embodiments, the first layer 334 and/or the second layer 336 may be formed from a polymeric material, a metallic material, and/or a composite material. In some embodiments, the first layer 334 and/or the second layer 336 may be formed from a substantially non-compliant material. In some other embodiments, the first layer 334 and/or the second layer 336 may be formed from an at least partially compliant material. In some embodiments, the first layer 334 and/or the second layer 336 may be formed form the same material as the inflatable annular member 332. In some embodiments, the first layer 334 and/or the second layer 336 may be formed form a different material as the inflatable annular member 332. In some embodiments, the first layer 334 and/or the second layer 336 may be permeable or semi-permeable. In some embodiments, the first layer 334 and/or the second layer may be non-permeable. Some suitable, but non-limiting, examples of materials for the sealing member 330, the inflatable annular member 332, the first layer 334, the mesh 335, the second layer 336, and/or the plurality of reinforcing fibers 338 are discussed below.

The alternative embodiment of FIG. 12 may have several features similar to those of other embodiments described herein. The implant 300 may comprise an expandable framework 310 configured to shift between a collapsed configuration and an expanded configuration. When the implant 300 is disposed within the lumen 120 of the delivery sheath 110, the expandable framework 310 may be held and/or disposed in the collapsed configuration, as shown in FIG. 14 for example. In some embodiments, the implant 300 may optionally include an occlusive element 320 disposed and/or positioned on, over, and/or around at least a portion of the expandable framework 310. In at least some embodiments, the occlusive element 320 may be secured to, attached to, and/or connected to the expandable framework 310. In some embodiments, the occlusive element 320 may be secured to, attached to, and/or connected to the expandable framework 310 at a plurality of discrete locations. In at least some embodiments, the expandable framework 310 may include a plurality of anchor members 312 extending therefrom, the plurality of anchor members 312 being configured to engage with the wall 54 of the main body 60 of the left atrial appendage 50. Some suitable, but non-limiting, examples of materials for the expandable framework 310, the plurality of anchor members 312, and the occlusive element 320 are discussed below.

The implant 300 may comprise the sealing member 330 spaced apart proximally from the expandable framework 310 by a flexible coupler 340. In at least some embodiments, the sealing member 330 may be secured to, attached to, and/or connected to the expandable framework 310 by the flexible coupler 340. In some embodiments, the flexible coupler 340 is tubular (e.g., a tubular member, a hollow tube, etc.) and includes a lumen extending therethrough. In some embodiments, the flexible coupler 340 may be formed by one or more filaments or sutures, one or more flexible members spaced apart from each other, a discontinuous flexible element having notches or cut-outs formed therein, a coiled member, or other suitable flexible structures. The sealing member 330 may be configured to shift between a delivery configuration and a deployed configuration. When the implant 300 and/or the sealing member 330 is disposed within the lumen 120 of the delivery sheath 110, the sealing member 330 may be held and/or disposed in the delivery configuration.

In some embodiments, the system 100 may include a core wire 130 releasably secured and/or releasably connected to the implant 300 at a distal end of the core wire 130. In some embodiments, the core wire 130 may be engaged with, releasably secured to, and/or releasably connected to the expandable framework 310 or the sealing member 330. In some embodiments, wherein the core wire 130 is engaged with, releasably secured to, and/or releasably connected to the expandable framework 310, the core wire 130 may pass through the sealing member 330. For example, the core wire 130 may pass through a self-sealing port and/or an aperture extending through the sealing member 330. In some embodiments, the core wire 130 may extend through the flexible coupler 340 to engage with the expandable framework 310. In some embodiments, the core wire 130 may engage with the sealing member 330 and/or the flexible coupler 340.

In some embodiments, the system 100 may include an inflation lumen 150 in fluid communication with the sealing member 330. The inflation lumen 150 may extend through the lumen 120 of the delivery sheath 110 to the sealing member 330. The sealing member 330 may be expandable under internal pressure exerted by an inflation fluid. In some embodiments, the inflation fluid may include a contrast agent for improved visualization under fluoroscopy. In some embodiments, the inflation fluid may be and/or include a hardening agent and/or a hardening or semi-hardening fluid. For example, the inflation fluid may include a biocompatible liquid such as saline, a hydropolymer, a hydrogel, or other suitable fluids. In at least some embodiments, an external shape of the sealing member 330 and/or the inflatable annular member 332 may be compliant, flexible, and/or adaptable to its surroundings. For example, the external shape of the sealing member 330 and/or the inflatable annular member 332 may shift and/or adapt to match the wall 54 and/or the ostium 56 of the left atrial appendage 50 disposed adjacent to the sealing member 330 and/or the inflatable annular member 332 upon implantation and thereby engage the wall 54 and/or the ostium 56 of the left atrial appendage 50 in a sealing manner.

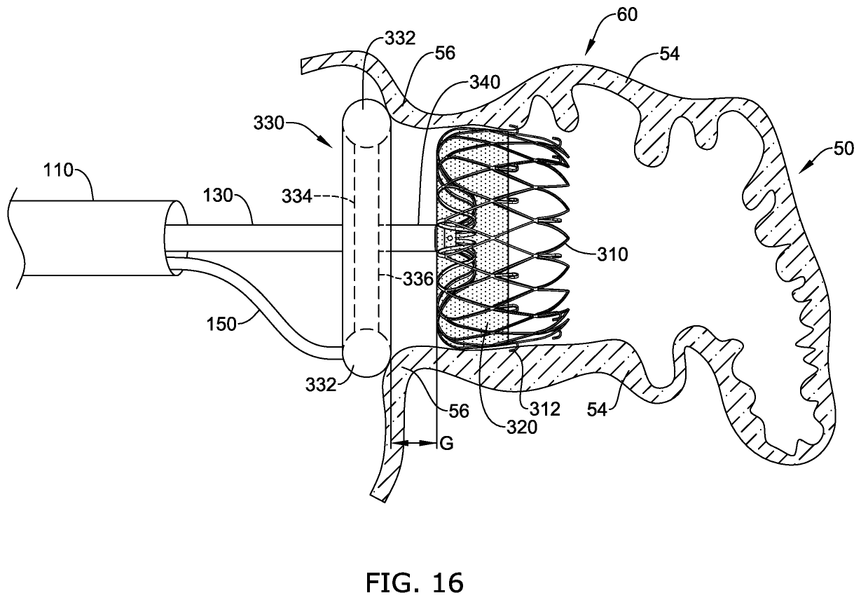

A method for occluding the left atrial appendage 50 may comprise advancing the implant 300 to the left atrial appendage 50. For example, the implant 300 may be advanced to the left atrial appendage within the lumen 120 of the delivery sheath 110. The method includes deploying the expandable framework 310 from the delivery sheath 110 within the left atrial appendage 50. The method further includes expanding and/or shifting the expandable framework 310 from the collapsed configuration to the expanded configuration within the left atrial appendage 50, as seen in FIG. 15 for example. In the expanded configuration, the expandable framework 310 may be urged into contact with, engaged with, and/or anchored to the wall 54 of the main body 60 of the left atrial appendage 50. Additionally, the method may include deploying the sealing member 330 proximate the ostium 56 of the left atrial appendage 50. In some embodiments, the sealing member 330 and/or the inflatable annular member 332 may be spaced apart proximally from the expandable framework 310 by a gap distance G, as seen in FIG. 16. The gap distance G may be generally understood as the axial distance between a proximal surface of the expandable framework 310 and a distal surface of the sealing member 330 and/or the inflatable annular member 332 measured generally parallel to a central longitudinal axis of the implant 300, the expandable framework 310, and/or the sealing member 330. In some embodiments, the gap distance G may be fixed. In some embodiments, the gap distance G may be variable. The expandable framework 310 and/or the plurality of anchor members 312 may function as an anchoring mechanism for the sealing member 330.

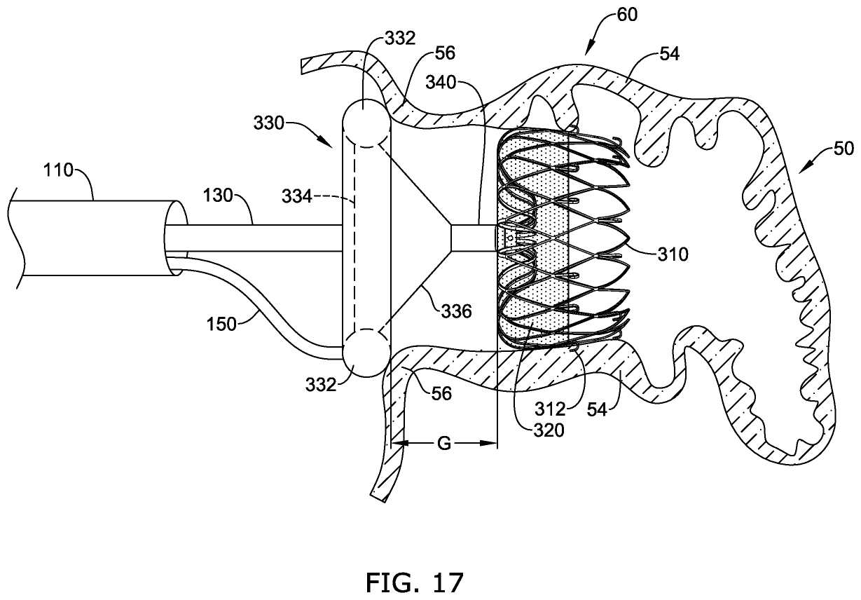

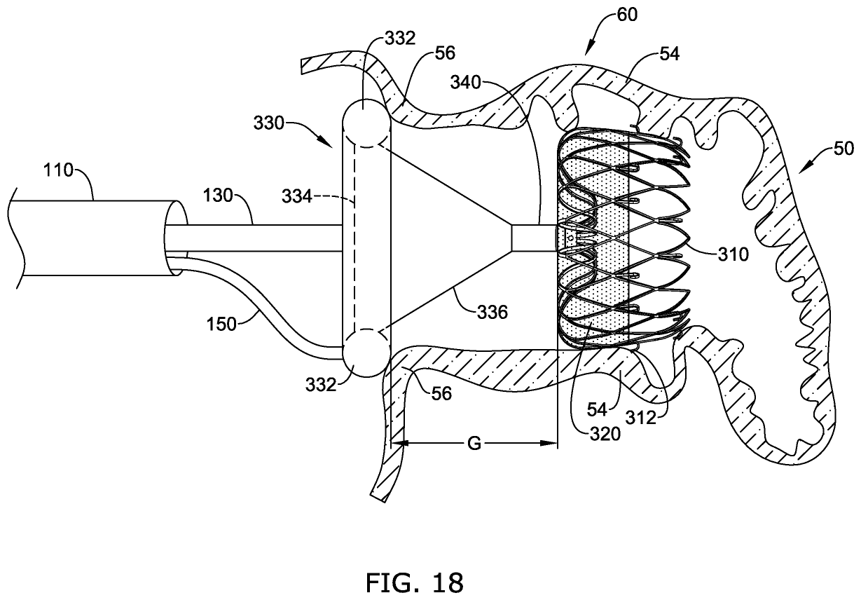

In some embodiments, the implant 300 may be configured to vary the gap distance G. For example, the second layer 336 may be secured to and/or connected to the flexible coupler 340. In some embodiments, the second layer 336 may be fixedly secured to and/or fixedly connected to a proximal end of the flexible coupler 340. In some embodiments, the second layer 336 may be non-compliant and/or non-stretchable relative to the inflatable annular member 332, wherein different degrees and/or magnitudes of inflation of the inflatable annular member 332 may determine adjustment of the gap distance G. For example, as the inflatable annular member 332 is inflated, the second layer 336 and/or the inflatable annular member 332 may be translated axially toward the expandable framework 310, thereby shortening and/or reducing the gap distance G. In one example, the gap distance G may be shortened by about 10%, about 20%, about 25%, about 30%, about 40%, about 50%, about 60%, about 75%, etc. from its initial deployment distance. In another example, the second layer 336 may be compliant and/or stretchable relative to the inflatable annular member 332, thereby permitting the expandable framework 310 to be placed deeper into the left atrial appendage 50. In some embodiments, as the inflatable annular member 332 is inflated, the second layer 336 and/or the inflatable annular member 332 may be translated axially away from the expandable framework 310, thereby increasing the gap distance G compared to a configuration where the second layer 336 is oriented and/or disposed perpendicular to the central longitudinal axis of the core wire 130 and/or the implant 300. In some embodiments, the implant 300 may include a translation member, disposed within and/or axially translatable relative to the core wire 130 for example, configured to axially translate the expandable framework 310 relative to the inflatable annular member 332. In some embodiments, the core wire 130 may be configured to axially translate the expandable framework 310 relative to the inflatable annular member 332. For example, the gap distance G may be increased by about 10%, about 30%, about 50%, about 70%, about 100%, about 150%, about 200%, etc. from its initial deployment distance, as seen in FIGS. 17 and 18. Additional arrangements and/or configurations are also contemplated. In at least some embodiments, as the inflatable annular member 332 is inflated the first layer 334 may remain substantially flat and/or non-compliant.

In some embodiments, the sealing member 330 may include an inflation port configured to accept and/or engage with the inflation lumen 150. In some embodiments, the inflation port may be a self-sealing port, and/or may include a hemostasis valve or other feature configured to seal the inflation port in the absence of a structure (e.g., the inflation lumen 150, etc.) disposed within and/or engaged with the inflation port. In at least some embodiments, the sealing member 330 and/or inflatable annular member 332 may be compliant and/or adaptable to its surroundings. As noted herein, the sealing member 330 and/or inflatable annular member 332 may shift and/or adapt to fit and/or match the contour of the wall 54 and/or the ostium 56 of the left atrial appendage 50 disposed adjacent to the sealing member 330 and/or inflatable annular member 332 upon implantation and thereby engage the wall 54 and/or the ostium 56 of the left atrial appendage 50 in a sealing manner. In at least some embodiments, the method may further include inflating at least a portion of the sealing member 330 (e.g., inflatable annular member 332) until the sealing member 330 and/or inflatable annular member 332 is capable of engaging the ostium 56 of the left atrial appendage 50 in a sealing manner.

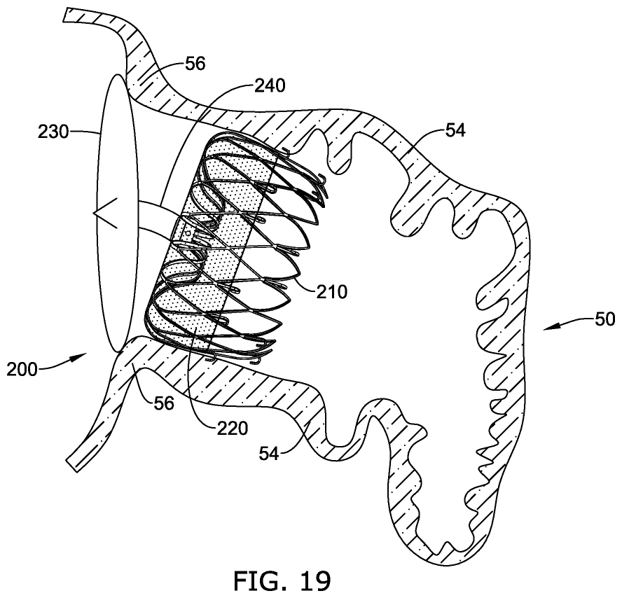

In some embodiments, the sealing member 230 may be oriented at an oblique angle to the central longitudinal axis of the expandable framework 210, as seen in FIG. 19. The flexible coupler 240 may permit the off-axis orientation of the sealing member 230 and the expandable framework 210 relative to each other, which may ease positioning, implantation, and/or sealing within an irregularly-shaped and/or oriented left atrial appendage 50. While not explicitly illustrated in the interest of brevity, the sealing member 330 of FIGS. 12-18 may also be oriented at an oblique angle to the central longitudinal axis of the expandable framework 310. The flexible coupler 340 may permit the off-axis orientation of the sealing member 330 and the expandable framework 310 relative to each other, which may ease positioning, implantation, and/or sealing within an irregularly-shaped and/or oriented left atrial appendage 50.

The materials that can be used for the various components of the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc. (and/or other systems or components disclosed herein) and the various elements thereof disclosed herein may include those commonly associated with medical devices. For simplicity purposes, the following discussion makes reference to the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc. However, this is not intended to limit the devices and methods described herein, as the discussion may be applied to other elements, members, components, or devices disclosed herein, such as, but not limited to, the plurality of anchor members 212/312, the inflatable annular member 332, the first layer 334, the second layer 336, the plurality of reinforcing fibers 338, etc. and/or elements or components thereof.

In some embodiments, the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc., and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc., and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc. to achieve the same result.

In some embodiments, a degree of Magnetic Resonance Imaging (MM) compatibility is imparted into the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc. For example, the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc., and/or components or portions thereof, may be made of a material that does not substantially distort the image and create substantial artifacts (e.g., gaps in the image). Certain ferromagnetic materials, for example, may not be suitable because they may create artifacts in an MM image. The system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc., or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc., and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the delivery sheath 110, the core wire 130, the inflation lumen 140/150, the expandable framework 210/310, the occlusive element 220/320, the sealing member 230/330, the flexible coupler 240/340, etc. disclosed herein may include a fabric material disposed over or within at least a portion of the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the delivery sheath 110, the core wire 130, the inflation lumen 140/150, the expandable framework 210/310, the occlusive element 220/320, the sealing member 230/330, the flexible coupler 240/340, etc. may include a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun-types. The type and denier of the yarn chosen may be selected in a manner which forms a biocompatible and implantable prosthesis and, more particularly, a vascular structure having desirable properties.

In some embodiments, the system 100, the delivery sheath 110, the core wire 130, the inflation lumen 140/150, the implant 200/300, the expandable framework 210/310, the occlusive element 220/320, the sealing member 230/330, the flexible coupler 240/340, the tapered member 250, etc. may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. An implant for occluding a left atrial appendage, comprising:
an expandable framework configured to shift between a collapsed configuration and an expanded configuration; and
a sealing member spaced apart proximally from the expandable framework by a gap distance.

2. The implant of claim 1, wherein the sealing member is connected to the expandable framework by a flexible coupler.

3. The implant of claim 2, wherein the flexible coupler is tubular.

4. The implant of claim 1, wherein the gap distance is variable.

5. The implant of claim 4, further comprising a tapered member configured to vary the gap distance.

6. The implant of claim 4, further comprising a threaded adjustment configured to vary the gap distance.

7. The implant of claim 6, wherein the threaded adjustment couples the sealing member to the expandable framework.

8. The implant of claim 1, wherein the sealing member includes an inflatable disk-shaped member.

9. The implant of claim 1, wherein the sealing member includes an inflatable annular member defining a central space.

10. The implant of claim 9, wherein the sealing member includes a first layer extending across the central space and a second layer extending across the central space.

11. The implant of claim 10, wherein the first layer is spaced apart from the second layer.

12. The implant of claim 10, wherein at least one of the first layer or the second layer includes a plurality of reinforcing fibers.

13. A system for occluding a left atrial appendage, comprising:
a delivery sheath having a lumen;
an implant for occluding the left atrial appendage, the implant comprising:
an expandable framework configured to shift between a collapsed configuration and an expanded configuration; and
a sealing member connected to and spaced apart proximally from the expandable framework by a flexible coupler; and
a core wire releasably secured to the implant.

14. The system of claim 13, further comprising an inflation lumen in fluid communication with the sealing member.

15. The system of claim 14, wherein the inflation lumen extends through the core wire.

16. A method for occluding a left atrial appendage, comprising:
advancing an implant to the left atrial appendage, the implant including:
an expandable framework configured to shift between a collapsed configuration and an expanded configuration; and
a sealing member spaced apart proximally from the expandable framework by a gap distance;
deploying the expandable framework within the left atrial appendage;
shifting the expandable framework into the expanded configuration within the left atrial appendage; and
deploying the sealing member proximate an ostium of the left atrial appendage.

17. The method of claim 16, further comprising:
inflating at least a portion of the sealing member until the sealing member is capable of engaging the ostium in a sealing manner.

18. The method of claim 16, further comprising:
adjusting the gap distance to position the sealing member against or within the ostium.

19. The method of claim 18, wherein the sealing member is oriented at an oblique angle to a central longitudinal axis of the expandable framework.

20. The method of claim 16, wherein the sealing member includes a mesh configured to promote endothelization.
